(19) United States
(12) Patent Application Publication
(10) Pub. No.: US 2026/0279491 A1
(43) Pub. Date: Sep. 17, 2026

(54) INFORMATION PROCESSING APPARATUS, INFORMATION PROCESSING METHOD, AND NON-TRANSITORY COMPUTER READABLE MEDIUM

(71) Applicant: KABUSHIKI KAISHA TOSHIBA, Kawasaki-shi (JP)

(72) Inventors: Sunao YOTSUTSUJI, Kawasaki Kanagawa (JP); Masaaki TAKADA, Kashiwa Chiba (JP)

(73) Assignee: KABUSHIKI KAISHA TOSHIBA, Kawasaki-shi (JP)

(21) Appl. No.: 19/549,559

(22) Filed: Feb. 25, 2026

(30) Foreign Application Priority Data
Mar. 12, 2025 (JP) 2025-039671

(51) Int. Cl. G16B 25/00; G16H 10/40; G16H 10/60

(52) U.S. Cl. CPC G16B 25/00; G16H 10/40; G16H 10/60

(57) ABSTRACT

An information processing apparatus includes processing circuitry configured to acquire a plurality of pieces of sample information for a plurality of samples where the sample information of a first sample among the plurality of samples includes first information, second information and third information. The first information is a measurement value of an expression level of a biomarker, the second information indicates a batch of samples measured under same or similar measurement conditions, and the third information indicates a state of a provider of the sample. The sample information of a second sample includes the first information and is missing at least one of the second information and the third information. The processing circuitry estimates the second information and the third information missing for the second sample. The processing circuitry calculates a batch dependent amount by which the expression level of the biomarker depends on the batch.

### CROSS REFERENCE TO RELATED APPLICATIONS

This application is based upon and claims the benefit of priority from the prior Japanese Patent Application No. 2025-039671, filed on Mar. 12, 2025, the entire contents of which are incorporated herein by reference.

### FIELD

Embodiments described herein relates to an information processing apparatus, an information processing method, and a non-transitory computer readable medium.

### BACKGROUND

Cancer has been the leading cause of death in Japan for a long period of time, and it is extremely important to detect cancer in the early stages. Development of a technique of estimating whether a person is affected with a specific disease including cancer has been promoted so far based on a plurality of pieces of biomarker information.

In estimation of whether a person is affected based on a measurement value of a gene expression level, negative influence on estimation performance due to a batch effect (error in the measurement value of the gene expression level caused by a measurement device and a measurement procedure different for each batch) becomes a problem. A plurality of batch effect correction methods has been proposed for post hoc analysis based on the measurement value of the gene expression level.

However, in estimation of whether a person is affected, there is a case where part of batch belonging information as to which batch a sample belongs to, and sample related information including whether the sample provider is affected (such as whether or not the sample provider is affected with a specific disease, age, and sex) may be missing. In such a case, it is impossible to appropriately correct the batch effect.

### BRIEF DESCRIPTION OF THE DRAWINGS

FIG. 1 is a block diagram schematically illustrating a configuration of a batch effect correction apparatus as an information processing apparatus according to the present embodiment.

FIGS. 2A to 2D each are views indicating an example of biomarker information, sample related information, batch belonging information, and measurement related information in tabular form.

FIGS. 3A and 3B each are views indicating an example of sample related missing information indicating a sample for which the sample related information is missing, and batch belonging missing information indicating a sample for which the batch belonging information is missing in tabular form.

FIG. 4 is a view indicating an example of the batch belonging information after missing portions are compensated for.

FIG. 5 is a view indicating an example of the sample related information after preprocessing.

FIG. 6 is a view indicating an example of the sample related information after missing portions are compensated for.

FIG. 7 is a view indicating the biomarker information for which the batch effect has been corrected.

FIG. 8 is a flowchart schematically indicating a procedure of processing to be performed by the information processing apparatus according to the embodiment of the present invention.

FIG. 9 is a view illustrating a hardware configuration of the information processing apparatus according to the present embodiment.

### DETAILED DESCRIPTION

According to one embodiment, an information processing apparatus includes processing circuitry configured to acquire a plurality of pieces of sample information for a plurality of samples where the sample information of a first sample among the plurality of samples includes first information, second information and third information. The first information is information regarding a measurement value of an expression level of a biomarker, the second information is information indicating a batch of samples measured under same or similar measurement conditions, and the third information is information regarding a state of a provider of the sample.

The sample information of a second sample includes the first information and is missing at least one of the second information and the third information.

The processing circuitry estimates the second information and the third information missing for the second sample based on the plurality of pieces of sample information.

The processing circuitry calculates a batch dependent amount by which the expression level of the biomarker depends on the batch, based on the plurality of pieces of sample information and the second information and the third information estimated for the second sample.

An embodiment of the present invention will be described below with reference to the drawings.

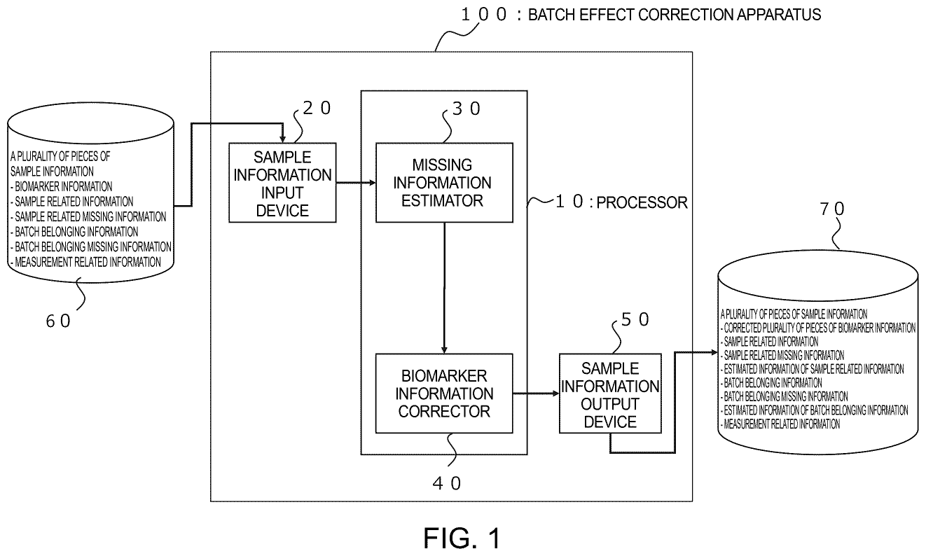

FIG. 1 is a block diagram schematically illustrating a configuration of a batch effect correction apparatus 100 as an information processing apparatus according to the present embodiment. The batch effect correction apparatus 100 includes a processor 10 which is processing circuitry, a sample information input device 20, a sample information output device 50, an input database 60, and an output database 70. The processor 10 includes a missing information estimator 30 and a biomarker information corrector 40.

The input database 60 stores information regarding a plurality of samples (sample information). Specifically, the input database 60 stores a biomarker table including biomarker information (first information) for each sample, a sample related table including sample related information (third information) for each sample, a batch belonging table including batch belonging information (second information) for each sample, and a measurement related table including measurement related information (fourth information) for each sample. However, in the sample related table and the batch belonging table, the sample related information and the batch belonging information are missing for some of the samples. Further, the input database 60 stores a sample related missing table including information indicating whether or not the sample related information is missing (sample related missing information) for each sample, and a batch belonging missing table including information indicating whether or not the batch belonging information is missing (batch belonging missing information) for each sample.

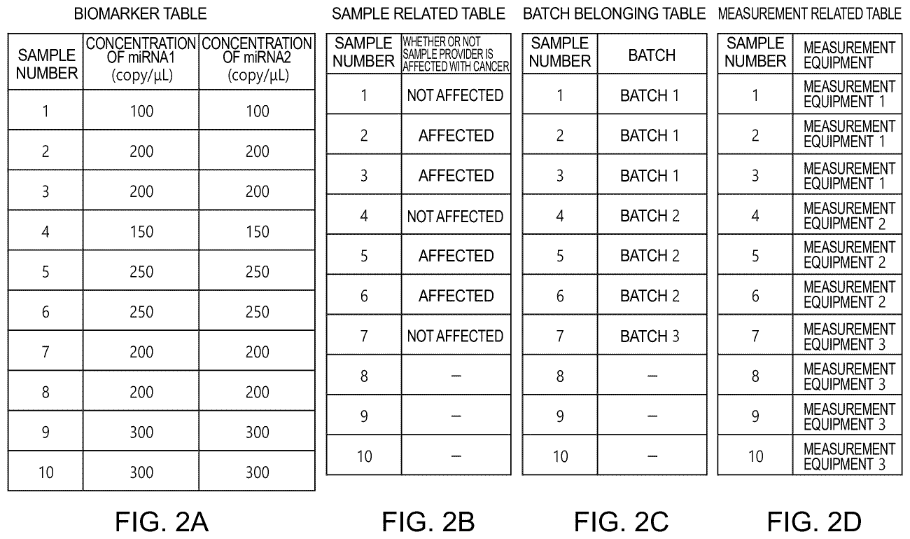

FIG. 2A indicates an example of the biomarker table. The biomarker table stores biomarker information (first information) for each sample. The biomarker information includes a measurement value of an expression level of a biomarker. In the present example, the biomarker information indicates measurement values of concentrations of miRNA1 and miRNA2. The measurement value of the concentration of miRNA is, for example, acquired using at least one of a PCR method, a LAMP method, a microarray method, a NanoString method, and a next-generation sequencing method. As one example, the measurement value of the concentration of miRNA is a measurement value in a case where serum or plasma is set as a sample to be measured. Each sample is identified by a sample number, and the measurement values of the concentrations of miRNA1 and miRNA2 are associated for each sample number. The biomarker information is not limited to these examples and may be other related information regarding miRNA1 and miRNA2 or may be information other than miRNA.

Note that while in the present example, the biomarker information indicates the measurement values of the expression levels of a plurality of biomarkers, the biomarker information may indicate only a measurement value of an expression level of one biomarker. Further, while in the present example, the biomarker information is a measurement value (concentrations of miRNA1 and miRNA2) of the biomarker itself, the biomarker information may be a value obtained by performing processing of any one of one or more calculations, standardization, whitening and log transformation on the measurement value one or more times. In this event, the processing may be performed through numerical calculation, statistical processing or using a machine learning method, and processing content may be adjusted in advance using other biomarker information. FIG. 2B indicates an example of the sample related table. The sample related table includes the sample related information (third information) for each sample. The sample related information is information representing a state of a collection source of the sample (sample provider). In the present example, a label (cancer label) indicating whether or not the sample provider is affected with cancer is indicated in association with the sample number. However, labels are missing for sample numbers 8 to 10.

The sample related information only requires to be information indicating the state regarding the sample provider and is not limited to whether or not the sample provider is affected with cancer. For example, the sample related information may be one or a plurality among whether the sample provider is affected with a disease (i.e., presence or absence of a disease), sex, age, a body height, a body weight, a past medical history, a medication history and an inspection value.

FIG. 2C indicates an example of the batch belonging table. The batch belonging table stores the batch belonging information (second information) for each sample. A label (batch label) indicating the batch to which the sample belongs is indicated in association with the sample number. However, the labels are missing for the sample numbers 8 to 10. The batch is a group of samples measured under the same or similar measurement conditions. In other words, a plurality of samples measured under the same or similar measurement conditions belongs to the same batch. In the present embodiment, in a case where a plurality of samples is measured with the same measurement equipment at the same time, these samples belong to the same batch. The similar measurement conditions indicate conditions in which influence on the measurement result statistically falls within an allowable range although content indicated in measurement related information which will be described later, such as settings of the measurement equipment and a measurement environment is not the same. For example, in a case where the measurement environment is an air temperature, even in a case where the air temperature is different, the measurement conditions can be regarded as similar measurement conditions in a case where a measurement error falls within the allowable range.

FIG. 2D indicates an example of the measurement related table. The measurement related table stores the measurement related information (fourth information) for each sample. A label (equipment label) indicating the measurement equipment with which the sample has been measured is indicated in association with the sample number. The equipment label is, for example, a model number of the measurement equipment. The measurement related information is not limited to the measurement equipment if the measurement related information indicates the measurement conditions of the sample.

For example, the measurement related information may include one or a plurality among settings of the measurement equipment, a measurement facility, a measurement environment, a person who has performed measurement, a measurement procedure, a measurement date and time, a model number of a measurement reagent, a manufacturing date and time of the measurement reagent, a sample collection date and time, and a sample collection procedure.

A sample for which the biomarker information, the sample related information, and the batch belonging information are present (are not missing) among a plurality of samples corresponds to a first sample, and a sample for which the biomarker information is present, but at least one of the sample related information and the batch belonging information is missing corresponds to a second sample.

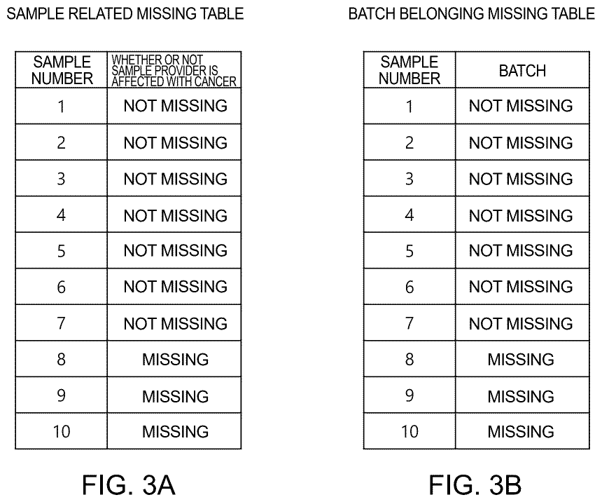

FIG. 3A indicates an example of the sample related missing table. The sample related missing table stores sample related missing information indicating whether or not the sample related information is missing for each sample. In the present example, information as to whether or not the cancer label of the sample provider is missing is indicated in association with the sample number.

FIG. 3B indicates an example of the batch belonging missing table. The batch belonging missing table stores batch belonging missing information indicating whether or not the batch belonging information is missing for each sample. In the present example, information as to whether or not the batch label regarding the sample is missing is indicated in association with the sample number.

It can be seen from FIG. 3A and FIG. 3B that the sample related information and the batch belonging information are missing for the samples (second samples) of the sample numbers 8 to 10 among the plurality of samples, and nothing is missing (all information is present) for other samples (first samples).

The sample information input device 20 reads a plurality of pieces of sample information from the input database 60 and provides the plurality of pieces of sample information to the missing information estimator 30. Each piece of sample information includes the biomarker information (first information), the sample related information (third information), the batch belonging information (second information), the measurement related information (fourth information), the sample related missing information, and the batch belonging missing information for the corresponding sample. In the present example, the sample information input device 20 reads information of the respective tables indicated in FIG. 2A to FIG. 2D and FIG. 3A and FIG. 3B and provides the information to the missing information estimator 30.

The missing information estimator 30 acquires a plurality of pieces of sample information read by the sample information input device 20 and estimates sample related information (whether or not the sample provider is affected with cancer) of the samples indicated as missing in the sample related missing information. Further, the missing information estimator 30 estimates the batch belonging information of the samples indicated as missing in the batch belonging missing information. The missing information estimator 30 can estimate only one of the sample related information and the batch belonging information or can estimate both the sample related information and the batch belonging information. Whether the missing information estimator 30 estimates one or both of the sample related information and the batch belonging information may be designated in advance by a user.

A specific example of processing of the missing information estimator 30 estimating the batch belonging information of the samples (second samples) will be described below.

As described above, in the batch belonging missing table indicated in FIG. 3B, the labels indicating the batches are missing for the samples of the sample numbers 8, 9, 10. Thus, the batches to which the samples of these sample numbers 8, 9, 10 belong are estimated.

First, the batch labels in the batch belonging table in FIG. 2C are made to correspond to the equipment labels in the measurement related table in FIG. 2D based on the sample numbers. As a result of this, a correspondence relationship in which measurement equipment 1 corresponds to a batch 1, measurement equipment 2 corresponds to a batch 2, and measurement equipment 3 corresponds to a batch 3 can be obtained. A model or a rule that makes measurement equipment X correspond to a batch X is generated based on this correspondence relationship.

It can be seen that the measurement equipment used for the samples of the sample numbers 8, 9, 10 is all the measurement equipment 3 as confirmed from the measurement related table in FIG. 2D. Thus, by inputting the equipment label of the measurement equipment 3 to the model generated earlier, the batch 3 can be obtained as estimated information (estimation result). In other words, it can be estimated that the samples of the sample numbers 8, 9, 10 belong to the batch 3. This estimated information is stored in missing portions of the batch belonging table in FIG. 2C. The table for which the missing portions of the batch belonging table have been compensated for in this manner will be referred to as a batch belonging table after the missing portions are compensated for.

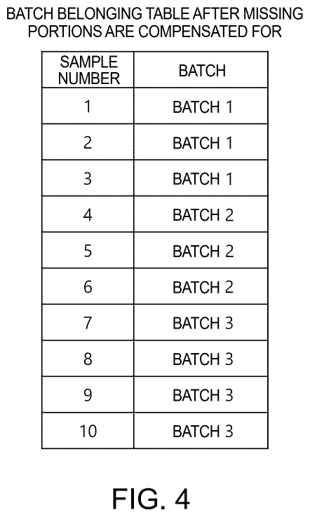

FIG. 4 indicates an example of the batch belonging table after the missing portions are compensated for. The estimated batch labels are stored in the missing portions of the sample numbers 8, 9, 10 in the batch belonging table in FIG. 2C.

Note that there can be also a case where measurement equipment of the sample for which the batch label is missing in the batch belonging table does not correspond to any of the measurement equipment 1 to 3. In this case, a new batch not included in the batch belonging information can be obtained as estimated information. For example, if the measurement equipment used for measurement is measurement equipment 4, there is a case where a batch 4 can be obtained as the estimated information.

On the other hand, for the sample for which the sample related information (whether or not the sample provider is affected with cancer) in the sample related table is missing, estimation is not performed here, and the sample related information is estimated at the same time as correction of the batch effect in a biomarker information corrector 40 which will be described later. Thus, in the present processing, information indicating “missing” is stored in the missing portions of the sample numbers 8, 9, 10 in the sample related table in FIG. 2B by the missing information estimator 30. A table in which information indicating “missing” is stored in the missing portions in the sample related table in this manner will be referred to as a sample related table after preprocessing.

Note that while in the present processing, estimation of the sample related information (whether or not the sample provider is affected with cancer) is omitted, the estimation may be performed using the biomarker information. In this case, it is only necessary to store the estimated sample related information in the missing portions of the sample related table. The table for which the missing portions have been compensated for in this manner will be referred to as a sample related table after the missing portions are compensated for.

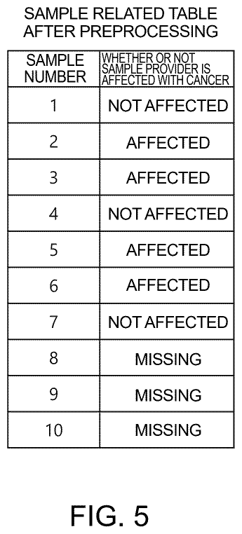

FIG. 5 indicates an example of the sample related table after preprocessing in which information (label) indicating missing is stored in the missing portions of the sample numbers 8, 9, 10 in the sample related table in FIG. 2B.

The biomarker information corrector 40 corrects the batch effect included in the biomarker information of each sample based on the biomarker table, the sample related table after preprocessing, the batch belonging table after the missing portions are compensated for, and the measurement related table.

More specifically, the biomarker information corrector 40 analyzes a relationship among an expression level of the biomarker, a batch, and a state of the sample provider and calculates an amount of the expression level of the biomarker depending on the batch (batch dependent amount). Then, the batch dependent amount is subtracted from the measurement value of the expression level of the biomarker. Then, the batch effect included in the biomarker information is corrected by subtracting the batch dependent amount from the measurement value of the expression level of the biomarker. The batch effect indicates an error in the measurement value of the expression level of the biomarker caused by measurement conditions (for example, the measurement device and the measurement procedure) different for each batch.

A specific example of processing of the biomarker information corrector 40 will be described below. The batch effect included in the biomarker information is corrected based on the biomarker table in FIG. 2A, the batch belonging table after the missing portions are compensated for in FIG. 4, and the sample related table after preprocessing in FIG. 5. Here, the batch effect is taken into account by Formula (1).

[Formula 1]

Y_{bjg}=α_{g}+X_{bj}β_{g}+γ_{bg} (1)
b: index indicating the batch
j: index indicating a position (rank order) of the sample number in the batch
g: index for identifying the biomarker
Y_{bjg}: variable indicating a value (measurement value) of a biomarker g of a sample number j of a batch b
X_{bj}: variable indicating sample related information of the sample number j of the batch b
α_{g}: variable indicating an average value of the biomarker g
β_{g}: variable indicating an effect of the biomarker g on sample related information
γ_{bg}: variable indicating a batch effect (amount of the expression level of the biomarker depending on the batch) of the batch b on the biomarker g

Note that the index j indicating the position of the sample number in the batch will be described. For example, in the batch belonging table in FIG. 2C, the index j is 1 in a case of the sample number 4. This is because the sample number 4 is a (first) sample having the smallest sample number in the batch 2.

Further, Y₂₁₁ is a value of the first biomarker (corresponding to miRNA1) having the first sample number (corresponding to the sample number 4 in the table) in the batch 2. Thus, Y₂₁₁=150.

In a similar manner, X₂₁ is a variable indicating the sample related information of the first sample number (sample number 4) in the batch 2. If a sample provider affected with cancer is defined as 1, and a sample provider not affected with cancer is defined as 0, X₂₁=0.

In the sample related table, information as to whether or not the sample provider is affected with cancer is missing for the sample numbers 8 to 10. Thus, the following variables can be acquired from FIG. 2A, FIG. 2B, and FIGS. 3 to 5.
(Y₁₁₁, Y₁₁₂, Y₁₂₁, Y₁₂₂, Y₁₃₁, Y₁₃₂, Y₂₁₁, Y₂₁₂, Y₂₂₁, Y₂₂₂, Y₂₃₁, Y₂₃₂, Y₃₁₁, Y₃₁₂, Y₃₂₁, Y₃₂₂, Y₃₃₁, Y₃₃₂, Y₃₄₁, Y₃₄₂) and (X₁₁, X₁₂, X₁₃, X₂₁, X₂₂, X₂₃, X₃₁)

On the other hand, other variables cannot be acquired and correspond to unobserved variables.
(X₃₂, X₃₃, X₃₄), α₁, α₂, β₁, β₂, γ₁₁, γ₁₂, γ₂₁, γ₂₂, γ₃₁, γ₃₂

In estimation of unobserved variables, a maximum likelihood estimation method or a Bayesian estimation method is used under appropriate assumption or approximation. For example, in the maximum likelihood estimation method, an EM algorithm, or the like, can be used. In the Bayesian estimation method, a Markov chain Monte Carlo sampling method, or the like, can be used.

As an example, the above-described unobserved variables can be estimated by repeating estimation of the batch effect, estimation of the cancer label, and the like. Here, the estimation of the batch effect is processing of estimating the batch effect (estimation of γ_{bg}) based on the biomarker table, the batch belonging table after the missing portions are compensated for, and the sample related table (including the tentative sample related information estimated in the middle of the processing).

The estimation of the cancer label is estimating portions missing in the sample related information (cancer label) (estimating X₃₂, X₃₃, X₃₄) based on the biomarker table, the batch belonging table after the missing portions are compensated for, the sample related table, and the estimated information (estimated value of γ_{bg}) of the batch effect.

In the present example, the following values can be obtained as a result of the estimation processing. X₃₂=0, X₃₃=X₃₄=1, α₁=α₂=100, β₁=β₂=100, γ₁₁=γ₁₂=0, γ₂₁=γ₂₂=50, γ₃₁=γ₃₂=100.

In the present example, the batch effect is corrected by subtracting γ_{bg} from Y_{bjg}. Specifically, the batch effect is corrected by subtracting the estimated value of the corresponding γ_{bg} from the measurement values (the concentration of miRNA1, the concentration of miRNA2) of the samples in the biomarker table in FIG. 2A.

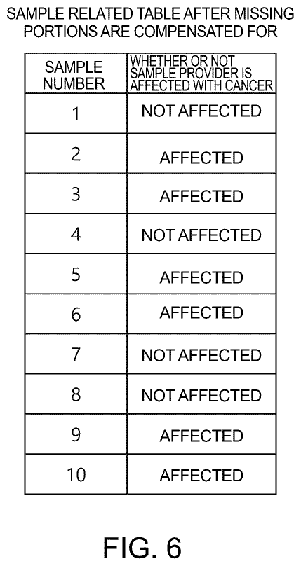

The estimated information of the cancer label may be stored in the missing portions in the sample related table based on the result of the present estimation processing. The sample related table in which the estimated information is stored corresponds to the sample related table after the missing portions are compensated for described above. An example of the sample related table after the missing portions are compensated for is indicated in FIG. 6.

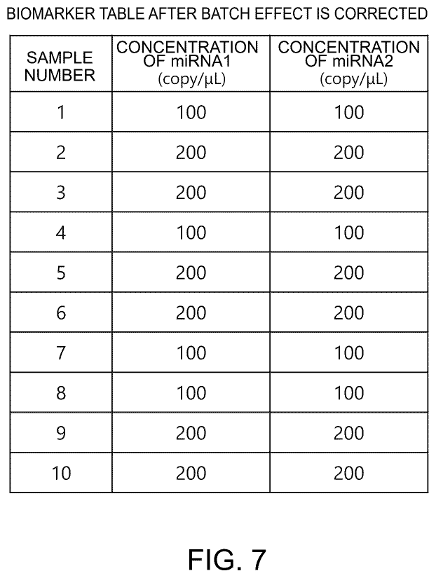

FIG. 7 indicates an example of a table (biomarker table after the batch effect is corrected) in which the biomarker information after the batch effect is corrected is stored. Compared with the biomarker table in FIG. 2A, each of the concentration of miRNA1 and the concentration of miRNA2 increases by 50 in the sample numbers 4 to 6 by influence of the correction. Further, each of the concentration of miRNA1 and the concentration of miRNA2 decreases by 100 in the sample numbers 7 to 10.

The sample information output device 50 acquires a plurality of pieces of sample e information including the corrected biomarker information acquired by the biomarker information corrector 40 and stores the plurality of pieces of sample information in the output database 70. In the present example, the following information is stored.
Biomarker table after the batch effect is corrected (see FIG. 7)
Sample related table (see FIG. 2B)
Batch belonging table (see FIG. 2C)
Measurement related table (see FIG. 2D)
Sample related missing table (see FIG. 3A)
Batch belonging missing table (see FIG. 3B)
Estimated information as to whether or not the sample provider is affected with cancer, estimated for the missing portions of the sample related table
Estimated information of the batch labels estimated for the missing portions in the batch belonging table

In place of or in addition to the sample related table (see FIG. 2B) and the estimated information as to whether or not the sample provider is affected with cancer, estimated for the missing portions of the sample related table, the sample related table after the missing portions are compensated for (see FIG. 6) may be output. Further, in place of or in addition to the batch belonging table (see FIG. 2C) and the estimated information of the batch labels, estimated for the missing portions in the batch belonging table, the batch belonging table after the missing portions are compensated for (see FIG. 4) may be output.

The sample information output device 50 is not limited to a device that writes the plurality of pieces of sample information including the corrected biomarker information in the output database 70 and may be other devices that output these pieces of information to the outside. For example, the sample information output device 50 may have a function as a display device such as a liquid crystal display device. In this case, the plurality of pieces of sample information including the corrected biomarker information may be displayed on a screen. Further, the sample information output device 50 may function as a communication device and can transmit the plurality of pieces of sample information including the corrected biomarker information to a terminal device possessed by the user or an external server.

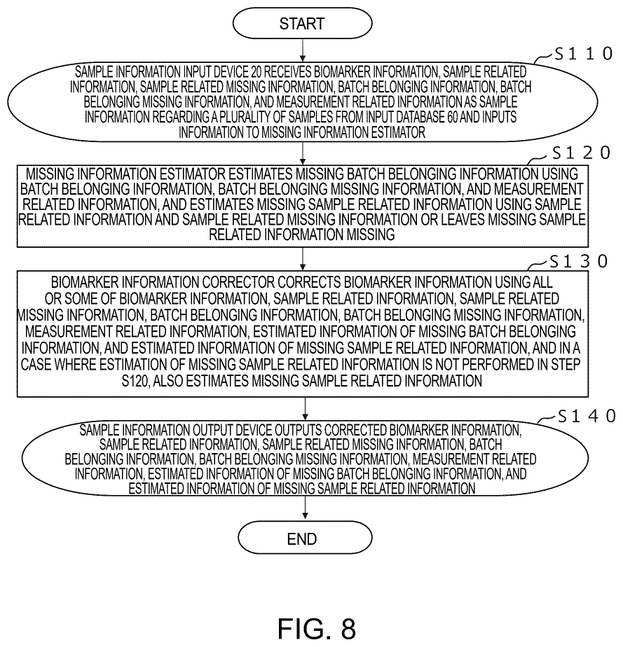

FIG. 8 is a flowchart schematically indicating a procedure of processing to be executed by the information processing apparatus according to the embodiment of the present invention. The procedure of the present processing is an example, and the estimation of the missing information and the correction of the batch effect do not necessarily have to be performed as indicated in the procedure.

In step S110, the sample information input device 20 receives a plurality of pieces of sample information from the input database 60 as inputs. These pieces of sample information include the biomarker information, the sample related information, the sample related missing information, the batch belonging information, the batch belonging missing information, and the measurement related information for the plurality of samples. At least one of the sample related information and the batch belonging information is missing for some of the samples (second samples). Whether or not the sample related information and the batch belonging information are present (whether or not there is a missing portion) for each sample is indicated by the sample related missing information and the batch belonging missing information.

In step S120, the missing information estimator 30 estimates the batch belonging information of the sample for which the batch belonging information is missing using the batch belonging information, the batch belonging missing information, and the measurement related information. Further, for the sample for which the sample related information is missing, processing (preprocessing) of setting a value of the sample related information as information indicating “missing” is performed. Note that in a case where the sample related information is missing, estimation may be also performed for the sample related information in a similar manner to the batch belonging information.

In step S130, the biomarker information corrector 40 corrects the batch effect included in the biomarker information based on the biomarker information, the sample related information, the sample related information subjected to the preprocessing, the batch belonging information, the estimated batch belonging information, and the measurement related information. In this event, estimation of the sample related information is also performed for the sample for which the sample related information is missing. However, in a case where the sample related information has been estimated in step S120, processing of estimating the sample related information of the sample for which the sample related information is missing in the present step S130 is not required.

In step S140, the sample information output device 50 outputs a plurality of pieces of sample information including the corrected biomarker information. The output plurality of pieces of sample information is stored in the output database 70.

Then, the processor 10 may perform any processing using the output database. For example, the processor 10 may generate an estimation model for estimating whether or not the sample provider is affected with cancer. Specifically, it is possible to generate a model that estimates whether or not the sample provider is affected with cancer from the biomarker information based on the corrected biomarker information, the sample related information, and the estimated sample related information. Then, by inputting biomarker information of a newly collected sample to the estimation model, it is possible to estimate whether the sample provider is affected with cancer.

As described above, according to the present embodiment, even in a case where the batch belonging information and the sample related information are missing for some of the samples, it is possible to correct the batch effect while estimating the batch belonging information and the sample related information. By this means, it can be expected to improve estimation accuracy as to whether or not the sample provider is affected with a specific disease such as cancer by the batch effect being appropriately corrected.

### (Modification 1)

The missing information estimator 30 can calculate the batch belonging information to be estimated for the sample for which the batch belonging information is missing or the sample related information to be estimated for the sample for which the sample related information is missing, as probability information.

For example, information indicating that, for a certain sample number, a probability of the “batch 3” is 60%, a probability of the “batch 1” is 20%, and a probability of the “batch 2” is 20% may be generated as estimated information of the batch belonging information. In other words, a probability that the sample belongs to each of a plurality of batches is calculated and can be utilized as the estimated information of the batch belonging information.

In a similar manner, in the sample related information, information indicating that, for a certain sample number, a probability that the sample provider is affected with cancer is 80%, and a probability that the sample provider is not affected with cancer is 20% may be calculated as estimated information of the sample related information. In other words, a probability that the sample is in each of a plurality of states is calculated and can be utilized as the estimated information of the sample related information.

For the sample for which the batch belonging information or the sample related information is missing in this manner, the batch belonging information or the sample related information of the sample is calculated as probability information. The biomarker information corrector 40 corrects the batch effect included in the biomarker information based on the calculated probability information. By this means, it can be expected to improve stability or accuracy of correction of the batch effect.

### (Modification 2)

The biomarker information does not have to be the measurement value itself of the expression level of the biomarker and may be a value obtained by performing processing of any one of any arithmetic correction, standardization, whitening, and log transformation on the measurement value one or more times. In this event, types or order of the processing can be freely set. By this means, it is possible to remove a measurement error other than the batch effect in advance. Further, it can be expected to make correction of the batch effect by the batch effect correction apparatus 100 efficient.

### (Modification 3)

While in the above-described embodiment, a case has been described where the batch belonging information and the sample related information are missing, the present invention can also address a case where there is a sample for which the biomarker information is missing. Missing of the biomarker information is considered to be often caused by a factor by measurement, and thus, the missing biomarker information can be compensated for using a typical missing value compensation method. For example, measurement values of the expression levels of the biomarkers of the samples belonging to the same batch are averaged, and the average value is utilized as the biomarker information of the sample for which the biomarker information is missing, belonging to the same batch. Alternatively, the missing data may be compensated for by utilizing correlation between the sample related information and the biomarker information. For example, regression analysis of the sample related information and the biomarker information is performed, and the missing biomarker information may be estimated using the obtained regression model.

### (Modification 4)

The processing of the batch effect correction apparatus 100 (processing of the flowchart in FIG. 8) may be repeatedly performed. Estimation accuracy of the missing information may be increased by correcting the batch effect. Thus, higher-accuracy batch effect correction can be expected by inputting the output information of the batch effect correction apparatus 100 to the batch effect correction apparatus 100 again and repeating the processing.

### (Modification 5)

The estimation for the sample for which the sample related information or the batch belonging information is missing can be performed not only by using the measurement related information but also by using one or a plurality of the biomarker information, the sample related information, and the batch belonging information.

For example, the missing batch belonging information can be estimated using the biomarker information. Specifically, a linear regression model that estimates the batch belonging information from the biomarker information is created based on the batch belonging information and the biomarker information of the sample for which the batch belonging information is not missing. The batch belonging information of the sample for which the batch belonging information is missing can be estimated by inputting the biomarker information of the sample to the linear regression model. Further, one of a non-linear regression model, a decision tree model, an NN model, and a logistic regression model can be used other than the linear regression model.

### (Hardware Configuration)

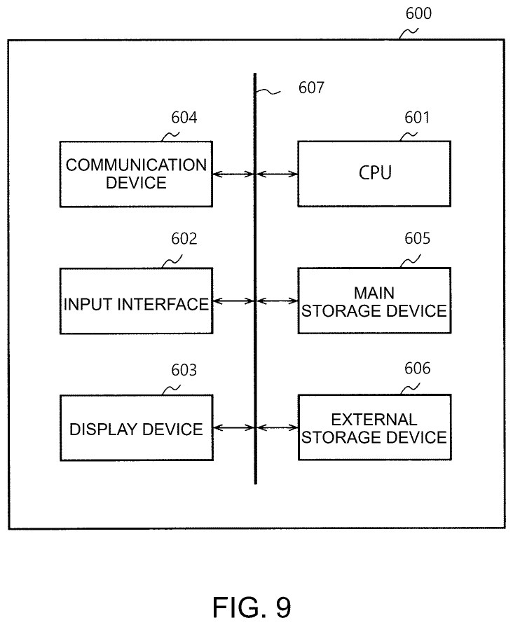

FIG. 9 illustrates a hardware configuration of the information processing device according to each embodiment. The information processing device is configured as a computer device 600. The computer device 600 includes a CPU 601, an input interface 602, a display device 603, a communication device 604, a main storage device 605, and an external storage device 606, and these components are mutually connected through a bus 607.

The CPU (central processing unit) 601 executes an information processing program as a computer program on the main storage device 605. The information processing program is a computer program configured to achieve each above-described functional composition of the present device. The information processing program may be achieved by a combination of a plurality of computer programs and scripts instead of one computer program. Each functional composition is achieved as the CPU 601 executes the information processing program.

The input interface 602 is a circuit for inputting, to the present device, an operation signal from an input device such as a keyboard, a mouse, or a touch panel. The input interface 602 corresponds to the input device in each embodiment.

The display device 603 displays data output from the present device. The display device 603 is, for example, a liquid crystal display (LCD), an organic electroluminescence display, a cathode-ray tube (CRT), or a plasma display (PDP) but is not limited thereto. Data output from the computer device 600 can be displayed on the display device 603. The display device 603 corresponds to the sample information output device 50.

The communication device 604 is a circuit for the present device to communicate with an external device in a wireless or wired manner. Data can be input from the external device through the communication device 604. The data input from the external device can be stored in the main storage device 605 or the external storage device 606. The communication device 604 corresponds to the sample information output device 50.

The main storage device 605 stores, for example, the information processing program, data necessary for execution of the information processing program, and data generated through execution of the information processing program. The information processing program is loaded and executed on the main storage device 605. The main storage device 605 is, for example, a RAM, a DRAM, or an SRAM but is not limited thereto. Each storage or database in the information processing device in each embodiment may be implemented on the main storage device 605.

The external storage device 606 stores, for example, the information processing program, data necessary for execution of the information processing program, and data generated through execution of the information processing program. The information processing program and the data are read onto the main storage device 605 at execution of the information processing program. The external storage device 606 is, for example, a hard disk, an optical disk, a flash memory, or a magnetic tape but is not limited thereto. Each storage or database in the information processing device in each embodiment may be implemented on the external storage device 606.

The information processing program may be installed on the computer device 600 in advance or may be stored in a storage medium such as a CD-ROM. Moreover, the information processing program in each embodiment may be uploaded on the Internet.

The present device may be configured as a single computer device 600 or may be configured as a system including a plurality of mutually connected computer devices 600.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

The embodiments as described before may be configured as below.
Clause 1. An information processing apparatus comprising a processing circuitry configured to:
   acquire a plurality of pieces of sample information for a plurality of samples where the sample information of a first sample among the plurality of samples includes first information, second information and third information,
      the first information being information regarding a measurement value of an expression level of a biomarker,
      the second information being information indicating a batch of samples measured under same or similar measurement conditions, and
      the third information being information regarding a state of a provider of the sample,
   the sample information of a second sample includes the first information and is missing at least one of the second information and the third information;
   estimate the second information and the third information missing for the second sample based on the plurality of pieces of sample information; and
   calculate a batch dependent amount by which the expression level of the biomarker depends on the batch, based on the plurality of pieces of sample information and the second information and the third information estimated for the second sample.
Clause 2. The information processing apparatus according to clause 1, wherein the processing circuitry is configured to analyze a relationship among the expression level of the biomarker, the batch, and the state of the provider to calculate the batch dependent amount.
Clause 3. The information processing apparatus according to clause 1 or 2, wherein the plurality of pieces of sample information includes fourth information indicating measurement conditions of the first sample and the second sample, and
   the processing circuitry is configured to estimate the second information missing for the second sample based on the fourth information of the first sample and the second sample and the second information of the first sample.
Clause 4. The information processing apparatus according to any one of clauses 1 to 3, wherein the processing circuitry is configured to estimate the second information missing for the second sample based on the first information of the first sample and the second sample and the second information of the first sample.
Clause 5. The information processing apparatus according to any one of clauses 1 to 4, wherein the processing circuitry is configured to estimate, for the second sample, a probability that the second sample belongs to each of a plurality of batches as the second information.
Clause 6. The information processing apparatus according to any one of clauses 1 to 5, wherein the processing circuitry is configured to estimate the third information missing for the second sample based on the first information of the first sample and the second sample and the third information of the first sample.
Clause 7. The information processing apparatus according to any one of clauses 1 to 6, wherein the processing circuitry is configured to estimate, for the second sample, a probability corresponding to each of a plurality of states as the third information.
Clause 8. The information processing apparatus according to any one of clauses 1 to 7, wherein the processing circuitry is configured to estimate the missing second information or the missing third information using any one of a linear regression model, a non-linear regression model, a decision tree model, an NN model, and a logistic regression model.
Clause 9. The information processing apparatus according to any one of clauses 1 to 8, wherein the processing circuitry is configured to correct the first information by subtracting the batch dependent amount from the first information regarding the plurality of samples.
Clause 10. The information processing apparatus according to clause 9, wherein the processing circuitry is configured to repeatedly perform processing of calculating the batch dependent amount based on the corrected first information and correcting the first information.
Clause 11. The information processing apparatus according to any one of clauses 1 to 10, wherein the first information is information based on a measurement value of a concentration of miRNA.
Clause 12. The information processing apparatus according to clause 11, wherein the first information is information based on the measurement value of the concentration of miRNA by at least one of a PCR method, a LAMP method, a microarray method, a NanoString method, and a next-generation sequencing method.
Clause 13. The information processing apparatus according to clause 12, wherein the measurement value of the concentration of miRNA is a measurement value in a case where serum or plasma is set as a sample to be measured.
Clause 14. The information processing apparatus according to any one of clauses 1 to 13, wherein the first information indicates a value obtained by applying any one of (i) correction based on an arbitrary calculation, (ii) standardization, (iii) whitening, and (iv) log transformation one or more times to the measurement value of the expression level of the biomarker.
Clause 15. The information processing apparatus according to any one of clauses 1 to 14, wherein, in a case where the first information is missing for at least one of the plurality of samples, the processing circuitry is configured to compensate for the missing first information based on the first information of the sample for which the first information is not missing.
Clause 16. The information processing apparatus according to clause 15, wherein the processing circuitry further compensates for the missing first information based on the third information of the sample for which the first information is not missing and the third information of the sample for which the first information is missing.
Clause 17. The information processing apparatus according to any one of clauses 1 to 16, wherein the third information indicates at least one of:
   presence or absence of a disease,
   sex,
   age,
   a body height,
   a body weight,
   a past medical history,
   a medication history, and
   an inspection value.
Clause 18. The information processing apparatus according to clause 17, wherein the disease is cancer.
Clause 19. The information processing apparatus according to clause 3, wherein the fourth information includes at least one of
   a model number of measurement equipment of the sample,
   settings of the measurement equipment,
   a measurement facility,
   a measurement environment,
   a person who has performed measurement,
   a measurement procedure,
   a measurement date and time,
   a model number of a measurement reagent,
   a manufacturing date and time of the measurement reagent,
   a sample collection date and time, and
   a sample collection procedure.
Clause 20. An information processing method executed by a computer, the method comprising:
   acquiring a plurality of pieces of sample information for a plurality of samples where the sample information of a first sample among the plurality of samples includes first information, second information and third information,
      the first information being information regarding a measurement value of an expression level of a biomarker,
      the second information being information indicating a batch of samples measured under same or similar measurement conditions, and
      the third information being information regarding a state of a provider of the sample,
   the sample information of a second sample includes the first information and is missing at least one of the second information and the third information;
   estimating the second information and the third information missing for the second sample based on the plurality of pieces of sample information; and
   calculating a batch dependent amount by which the expression level of the biomarker depends on the batch, based on the plurality of pieces of sample information and the second information and the third information estimated for the second sample.
Clause 21. A non-transitory computer readable medium storing a computer program for causing a computer to execute:
   acquiring a plurality of pieces of sample information for a plurality of samples where the sample information of a first sample among the plurality of samples includes first information, second information and third information,
      the first information being information regarding a measurement value of an expression level of a biomarker,
      the second information being information indicating a batch of samples measured under same or similar measurement conditions, and
      the third information being information regarding a state of a provider of the sample,
      the sample information of a second sample includes the first information and is missing at least one of the second information and the third information;
   estimating the second information and the third information missing for the second sample based on the plurality of pieces of sample information; and
   calculating a batch dependent amount by which the expression level of the biomarker depends on the batch, based on the plurality of pieces of sample information and the second information and the third information estimated for the second sample.

1. An information processing apparatus comprising processing circuitry configured to:
acquire a plurality of pieces of sample information for a plurality of samples where the sample information of a first sample among the plurality of samples includes first information, second information and third information,
the first information being information regarding a measurement value of an expression level of a biomarker,
the second information being information indicating a batch of samples measured under same or similar measurement conditions, and
the third information being information regarding a state of a provider of the sample,
the sample information of a second sample includes the first information and is missing at least one of the second information and the third information;
estimate the second information and the third information missing for the second sample based on the plurality of pieces of sample information; and
calculate a batch dependent amount by which the expression level of the biomarker depends on the batch, based on the plurality of pieces of sample information and the second information and the third information estimated for the second sample.

2. The information processing apparatus according to claim 1, wherein the processing circuitry is configured to analyze a relationship among the expression level of the biomarker, the batch, and the state of the provider to calculate the batch dependent amount.

3. The information processing apparatus according to claim 1, wherein the plurality of pieces of sample information includes fourth information indicating measurement conditions of the first sample and the second sample, and
the processing circuitry is configured to estimate the second information missing for the second sample based on the fourth information of the first sample and the second sample and the second information of the first sample.

4. The information processing apparatus according to claim 1, wherein the processing circuitry is configured to estimate the second information missing for the second sample based on the first information of the first sample and the second sample and the second information of the first sample.

5. The information processing apparatus according to claim 1, wherein the processing circuitry is configured to estimate, for the second sample, a probability that the second sample belongs to each of a plurality of batches as the second information.

6. The information processing apparatus according to claim 1, wherein the processing circuitry is configured to estimate the third information missing for the second sample based on the first information of the first sample and the second sample and the third information of the first sample.

7. The information processing apparatus according to claim 1, wherein the processing circuitry is configured to estimate, for the second sample, a probability corresponding to each of a plurality of states as the third information.

8. The information processing apparatus according to claim 1, wherein the processing circuitry is configured to estimate the missing second information or the missing third information using any one of a linear regression model, a non-linear regression model, a decision tree model, an NN model, and a logistic regression model.

9. The information processing apparatus according to claim 1, wherein the processing circuitry is configured to correct the first information by subtracting the batch dependent amount from the first information regarding the plurality of samples.

10. The information processing apparatus according to claim 9, wherein the processing circuitry is configured to repeatedly perform processing of calculating the batch dependent amount based on the corrected first information and correcting the first information.

11. The information processing apparatus according to claim 1, wherein the first information is information based on a measurement value of a concentration of miRNA.

12. The information processing apparatus according to claim 11, wherein the first information is information based on the measurement value of the concentration of miRNA by at least one of a PCR method, a LAMP method, a microarray method, a NanoString method, and a next-generation sequencing method.

13. The information processing apparatus according to claim 12, wherein the measurement value of the concentration of miRNA is a measurement value in a case where serum or plasma is set as a sample to be measured.

14. The information processing apparatus according to claim 1, wherein the first information indicates a value obtained by applying any one of (i) correction based on an arbitrary calculation, (ii) standardization, (iii) whitening, and (iv) log transformation one or more times to the measurement value of the expression level of the biomarker.

15. The information processing apparatus according to claim 1, wherein, in a case where the first information is missing for at least one of the plurality of samples, the processing circuitry is configured to compensate for the missing first information based on the first information of the sample for which the first information is not missing.

16. The information processing apparatus according to claim 15, wherein the processing circuitry further compensates for the missing first information based on the third information of the sample for which the first information is not missing and the third information of the sample for which the first information is missing.

17. The information processing apparatus according to claim 1, wherein the third information indicates at least one of:
presence or absence of a disease,
sex,
age,
a body height,
a body weight,
a past medical history,
a medication history, and
an inspection value.

18. The information processing apparatus according to claim 17, wherein the disease is cancer.

19. The information processing apparatus according to claim 3, wherein the fourth information includes at least one of
a model number of measurement equipment of the sample,
settings of the measurement equipment,
a measurement facility,
a measurement environment,
a person who has performed measurement,
a measurement procedure,
a measurement date and time,
a model number of a measurement reagent,
a manufacturing date and time of the measurement reagent,
a sample collection date and time, and
a sample collection procedure.

20. An information processing method executed by a computer, the method comprising:
acquiring a plurality of pieces of sample information for a plurality of samples where the sample information of a first sample among the plurality of samples includes first information, second information and third information,
the first information being information regarding measurement value of an expression level of a biomarker,
the second information being information indicating a batch of samples measured under same or similar measurement conditions, and
the third information being information regarding a state of a provider of the sample,
the sample information of a second sample includes the first information and is missing at least one of the second information and the third information;
estimating the second information and the third information missing for the second sample based on the plurality of pieces of sample information; and
calculating a batch dependent amount by which the expression level of the biomarker depends on the batch, based on the plurality of pieces of sample information and the second information and the third information estimated for the second sample.

21. A non-transitory computer readable medium storing a computer program for causing a computer to execute:
acquiring a plurality of pieces of sample information for a plurality of samples where the sample information of a first sample among the plurality of samples includes first information, second information and third information,
the first information being information regarding a measurement value of an expression level of a biomarker,
the second information being information indicating a batch of samples measured under same or similar measurement conditions, and
the third information being information regarding a state of a provider of the sample,
the sample information of a second sample includes the first information and is missing at least one of the second information and the third information;
estimating the second information and the third information missing for the second sample based on the plurality of pieces of sample information; and
calculating a batch dependent amount by which the expression level of the biomarker depends on the batch, based on the plurality of pieces of sample information and the second information and the third information estimated for the second sample.
